(19) United States
(12) Patent Application Publication
(10) Pub. No.: US 2012/0296434 A1
(43) Pub. Date: Nov. 22, 2012

(54) KNEE PROSTHESIS

(76) Inventor: Pranesh Kumar, Rotorua (NZ)

(21) Appl. No.: 13/514,239

(22) PCT Filed: Oct. 21, 2010
(86) PCT No.: PCT/NZ2010/000211

§ 371 Date: Jun. 6, 2012

Related U.S. Application Data
(60) Provisional application No. 61/253,907, filed on Oct. 22, 2009.

(51) Int. Cl. A61F 2/30; A61B 17/84

(52) U.S. Cl. 623/18.11; 606/300

(57) ABSTRACT

Osteoarthritis (OA) is the most common disease affecting human joints. Mechanical stress through the joint is one of the most important independent etiological factors. The present invention provides a prosthesis that bypasses some of the stress from the joint without destroying the joint surface. It allows full range of joint movement, while sharing the load with the physiological joint, thereby maintaining the viability of the physiological joint surface.

### STATEMENT OF CORRESPONDING APPLICATIONS

This application is based on U.S. Provisional Patent Application No. 61/253,907 the entire contents of which are incorporated herein by reference.

### TECHNICAL FIELD

The present invention relates to a prosthetic device which is surgically implantable into a body joint to support the joint.

### BACKGROUND ART

Osteoarthritis (OA) is the most common disease affecting human joints. It is second only to cardiovascular disease as the cause of chronic disability in adults. Worldwide, billions of dollars are spent annually for its treatment and for the lost days in work.

OA is widely considered to be a degenerative joint disease and more than 50% of individuals above the age of 65 years have clinical evidence of OA. Nevertheless, OA cannot be described as a simple consequence of aging. Epidemiological studies have shown a strong correlation of OA with obesity, physical sports and occupation. Moreover, mechanical stress through the joint has been suggested as one of the most important independent etiological factors.

While physiological stress is needed for cartilage and bone sustenance and repair, excessive stress through joint surface leads to initiation and progression of OA. Prolonged high stress and excessive impulsive stress are detrimental to cartilage viability, whereas, repetitive physiological stress is beneficial for cartilage health.

Patients with OA generally present with pain, stiffness and deformity of the joint. Present treatment protocols are mainly symptomatic treatment. Initial management of most patients includes changes in lifestyle, Non-Steroidal Anti-Inflammatory Drugs (NSAIDs), analgesics, physical therapy, bracing and ambulatory aids. Surgical treatment is indicated only when consecutive treatment fails to improve the symptoms.

With particular reference to the knee common surgical options include arthroscopic debridement, high tibial osteotomy (HTO), and unicompartmental or tricompartmental knee replacement. In general, present forms of joint replacement surgery completely sacrifice the natural joint and only provide limited symptom relief and restricted mobility. Further, the lifespan of the replaced joint is also limited. None of the presently available treatment methods change the natural progress of the disease.

It is known to provide knee implants. One example of such a device is disclosed in United States Patent Publication No. 2008/0275561 A1 to Exploramed NC4, Inc. The patent discloses various implants used for absorbing energy between body parts, and in particular knee joints.

The implants disclosed in the Exploramed patent are intended to absorb energy when the knee is extended (e.g. the leg is straight). That energy is absorbed by an energy manipulator such as a spring or elastomeric material. The energy is subsequently distributed into the localised knee area on flexion of the knee (e.g. bending of the leg). However, the configurations of the implants of the Exploramed patent only rely on the native knee joint to provide the range of motion for knee movement. That is, the ends of the tibia and femur bones continue to provide articulation of the knee. This limits effectiveness of the disclosed devices.

Yet a further limitation of the Exploramed patent implants is that these do not promote regeneration of native cartilage or the femur/tibia bones. This is due to native knee joint providing the motion, which may continue to aggravate the diseased joint.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

It is an object of the present invention to provide an improved knee prosthesis and method of employing same, or to at least provide the public or medical profession with a useful choice.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### DISCLOSURE OF THE INVENTION

According to one aspect of the present invention, there is provided a prosthesis for insertion into a joint, including

a first plate configured for fixing to a first bone forming part of the joint,

a second plate configured for fixing to a second bone forming part of the joint,

bearing surfaces associated with the first plate and the second plate,

characterised in that

the bearing surfaces are shaped to cooperate with each other to guide movement of the second bone relative to the first bone through a desired range of motion.

According to another aspect of the present invention, there is provided a method of implanting a prosthesis in to a joint, including the following steps in any order:
(a) aligning a first plate including a bearing surface with a first bone forming part of the joint;
(b) securing a first plate to the first bone;
(c) securing the second plate to the second bone;
(d) aligning a second plate including a bearing surface with a second bone forming part of the joint;
wherein the respective bearing surfaces of the first plate and the second plate contact each other;
characterised by the step of
(e) shaping the bearing surfaces to co-operate with each other to guide movement of the second bone relative to the first bone through a desired range of motion.

A prosthesis kit, including
(a) a first plate configured for fixing to a first bone forming part of a joint;
(b) a second plate configured for fixing to a second bone forming part of the joint;
wherein each of the first plate and second plate have bearing surfaces;
characterised in that the bearing surfaces are shaped to co-operate with each other when the prosthesis is fitted into the joint so as to guide movement of the second bone relative to the first bone through a desired range of motion.

According to another aspect of the present invention, there is provided a fastener for fixing a prosthetic structure to bone, including
a body with a length,
a first point end,
a second end distal to the first end
wherein the second end is configured to facilitate inserting the fastener into bone
characterised in that
the body has a section with a generally triangular cross section.

The present invention aims to alter the etio-pathogenesis of the disease process, by providing a mechanism to partially bypass the stresses experienced by a joint surface. In a preferred embodiment this is achieved by sharing the load with the native joint surface. The implant of the invention assists the joint to bear the prolonged constant high stress and excessive impulsive stresses that are detrimental to joint physiology, while maintaining some physiological stress to be transferred through the native joint surface.

It should be appreciated that the term “desired range of motion” refers to the preferred motion path as well as the angle of movement. That is, the joint is guided to move as it is physiologically designed to do so, and preferably with a full range of normal movement eg maximum extension/flexion, and/or pronation/supination.

Importantly, the present prosthesis may provide a full range of motion of the treated joint. In doing so, it may protect the native bones forming the joint, while potentially providing symptomatic relief, improved joint recovery and improved function. The present prosthesis can also be applied using a relatively smaller operation than total joint replacements, or in the case of a knee prosthesis the High Tibial Osteotomy (HTO).

In a preferred embodiment the present invention is intended for use in joints including knees, elbows, ankles, fingers, shoulders, wrists, or hips. Various exemplary joints including embodiments will be discussed herein. However the discussion of these embodiments should not be seen as limiting and alternatives are envisaged.

Throughout the present specification reference will be made to first bone and second bone of a joint. These terms should be given their ordinary meaning as would be known to those skilled in the art. For instance, in the embodiment of a knee prosthesis the first bone is the femur while the second bone is the tibia. In a hip prosthesis the first bone is the pelvis while the second bone is the femur.

Certain joints may include third bones such as the radius of the elbow. Embodiments of the present to account for such joints are discussed below.

Throughout the present specification the term first plate or second plate should be understood as meaning components having a width and which are configured for fixing to a first or second bone.

In a preferred embodiment the first and/or second plates are shaped so as to correspond to the bone to which each will be attached. This enables force placed on the prosthesis to be distributed more evenly with respect to the joint, rather than creating points of concentrated pressure. The prosthesis may also be more compact and better suited for insertion into the joint.

The present invention may also include more than two plates. This will depend on the particular joint with which a prosthesis is used. For instance in an elbow or shoulder joint, a third plate(and even fourth plate), could be secured to bones forming the joint so as to facilitate movement of the joint. In the case of an elbow joint the second plate may be secured to the ulna and the third plate secured to the radius. Each of the plates may have a bearing surface that cooperates with a corresponding bearing surface on one or more plates secured to the humerus.

It is also envisaged that two pairs of plates could be used. For instance, in a knee joint, a pair of first and second plates could be attached to the lateral margins of a knee joint and a second pair of plates is fixed to the medial margins of a knee joint. In this embodiment, the bearing surfaces of each pair of first and second plates are shaped so as to conform to, and/or mimic, the shape of condyles of bones forming the knee joint.

In a preferred embodiment, the plates may be shaped and/or otherwise configured so as to facilitate its insertion into a joint while accommodating the joint's native ligaments.

For instance, a plate may have a spiral shaped aperture at an edge. This will allow the plate to be twisted around the ligament before being secured into position to the native bone.

Alternatively, each plate may be provided in two or more components which are releasably securable to each other. In use, the first component of the plate is positioned relative to the joint so that ligament extends through an aperture in the plate. A second component of the plate is positioned relative to the first component and secured thereto using fastener mechanisms.

These features held to secure the plates in position without the necessity of cutting the native ligaments. This may promote patient rehabilitation.

Throughout the present specification reference to the term bearing surface should be understood as meaning a component providing a point of contact between the first and second plates. That is, weight or force applied to the joint is not substantially transferred to the native joint articulating surface. Rather, the plates transfer the force to the antero-lateral and/or antero-medial margins of native bones. This should become clearer from the following description.

The bearing surfaces also act as articulating surfaces, meaning that they facilitate the first plate and second plate, and therefore bodies of the joints, moving with respect to each other. This may be provided by the bearing surfaces having low frictional coefficients so that they can slide relative to each other. Alternatively, ball bearings or other components may be provided between the bearing surfaces so as to allow these to move with respect to each other. This is useful is providing a joint with a desired range of motion.

In the preferred embodiment the bearing surfaces cooperate so as to guide the movement of the bones forming the joint with respect to each other. This is achieved by providing at least one of the bearing surfaces with a shape corresponding to the desired range of motion.

In a particularly preferred embodiment, at least one of the bearing surfaces may have a shape corresponding to an articulating surface of a natural joint. For instance, a bearing surface of a knee prosthesis may be shaped so as to conform to, or mimic, the condyles of the femur. This bearing surface is a complex shape, having a series of involute midpoints generally falling on a spiral. The cooperating bearing surface is shaped to correspond to the condyles of the tibia.

Alternative embodiments of the bearing surfaces will be discussed in more detail below by reference to different embodiments of prosthesis according to the present invention.

It is also envisaged that the bearing surfaces can be shaped so as to provide a desired range of motion, other than that of native joint.

In yet a further embodiment, the bearing surfaces may be shaped so as to provide a range of motion for the joint corresponding to that of a native joint, yet have a shape which does not correspond to the articulating surfaces of that native joint. For instance, in an embodiment of an ankle joint, bearing surfaces of first and second plates define a range of motion corresponding to an arc of motion of the native joint, yet have shapes that do not correspond to the articulating surfaces of the ankle joint.

Accordingly, the foregoing should not be seen as limiting.

These aspects of the present invention should become clearer from the following description.

In a preferred embodiment, the prosthesis according to the present invention are configured to maintain separation of the native bones forming a joint. This allows the bearing surfaces to facilitate the range of motion for the native joint surfaces, yet may minimise aggravation to those surfaces. That is, the bearing surfaces act as and provide, a track and guide for the bones to move without relying on the native joint surfaces. Note that the movement of the bones occurs by (or over) the bearing surfaces touching each other, rather than the native joint surfaces. These aspects of the present invention should become clearer from the following discussion of the preferred embodiments of the present invention.

Various embodiments are envisaged for the track and guide aspects of the bearing surfaces. For instance, bearing surfaces may be members and channels/grooves. Alternatively, a track may be a channel having a curve within which an elongate bearing surface having a corresponding curve can move.

Alternatively, ball and socket type arrangements are envisaged.

Yet a further embodiment of a track and guide envisaged as being within the scope of the present invention is a recess having a shelf or lip. Such an arrangement provides a bearing surface having a shape corresponding to the desired range of motion. A corresponding bearing surface cooperates with the recess and shelf/lip.

Accordingly, the foregoing should not be seen as limiting.

These aspects of the present invention should become clearer by reference to the following description of the preferred embodiments.

Throughout the present specification reference to the term “range of motion” should now be understood as meaning the distance and direction of movement of two or more bones forming a joint with respect to each other.

In a preferred embodiment the desired range of motion is a normal range of motion of a joint. That range of motion will vary between different types of joints according to each joints' native characteristics. For instance, in a knee prosthesis the present invention will allow the femur and tibia bones to move with respect to each other through a normal range of flexion and extension. The prosthesis enables the bones to rotate so as to accommodate locking of the knee at extension.

In an alternate embodiment such as an elbow joint, the prosthesis can provide flexion and extension of the joint according to normal movement of the ulna and humerus. The prosthesis also facilitates rotational movement of the radius that occurs during pronation or supination of the forearm.

However the foregoing should not be seen as limiting as alternatives are envisaged including those where the prosthesis provides a range of motion less than a full range for a native joint. This may be beneficial where joint mobility is to be restricted to account for a medical condition or limitations of another joint/limb.

In a preferred embodiment, the prosthesis according to the present invention are configured so as to transfer some of the stress to which the joint is exposed into cartilage of the joint. This may be achieved by relative spacing or interaction of the first plate and second plate, and/or their respective bearing surfaces.

Alternatively, a deformable component may be utilised. The deformable component allows movement of the first plate and second plate towards each other. However, the deformable component maintains sufficient separation of the native bones forming the joint such that these do not touch each other and articulation of the joint occurs via the bearing surfaces.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages the construction and use of alternatives, of which the following gives examples only.

### BRIEF DESCRIPTION OF THE DRAWINGS

Further aspects of the present invention will become apparent from the ensuing description which is given by way of example only and with reference to the accompanying drawings in which:

FIG. 1 is a back view of the prosthesis in situ, attached to a knee joint.

FIG. 2 is a rear perspective view of the joint and prosthesis shown in Figure

FIG. 3 is a front view of the prosthesis and knee joint shown in with cross rods inserted.

FIG. 4 is a rear perspective view of the prosthesis shown without the joint.

FIG. 5A is an exploded perspective view of a preferred embodiment of a femoral component of the prosthesis.

FIG. 5B is an exploded perspective view of an alternate embodiment of a femoral component of the prosthesis.

FIG. 6A is an exploded perspective view of a preferred embodiment of a tibial component of the prosthesis.

FIG. 6B is an exploded perspective view of an alternate embodiment of a tibial component of the prosthesis.

FIG. 7 is a view of a femoral component and a tibial component showing the mounting features.

FIG. 8A is a perspective view of a fastener pin.

FIG. 8B is a perspective view of an alternate embodiment of a fastener pin.

FIG. 9A is a back perspective view of a femur bearing surface.

FIG. 9B is a side view of FIG. 9A.

FIG. 9C is a bottom view of FIGS. 9A and 9B.

FIG. 10 is a front view of an elbow in extension with a prosthesis according to the present invention.

FIG. 11 is a side view of an elbow in flexion with a prosthesis according to the present invention.

FIG. 12 is a side view of a finger in extension with the prosthesis according to the present invention.

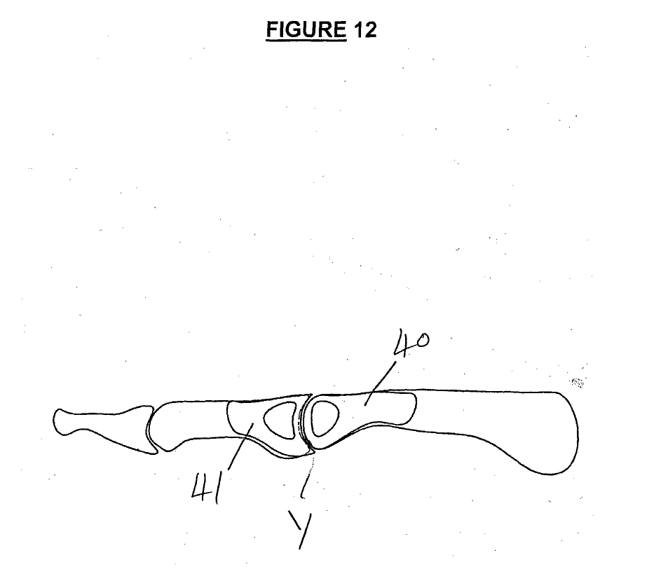

FIG. 13 is a front view of FIG. 12.

FIG. 14 is a front view of a hip joint with a prosthesis according to the present invention.

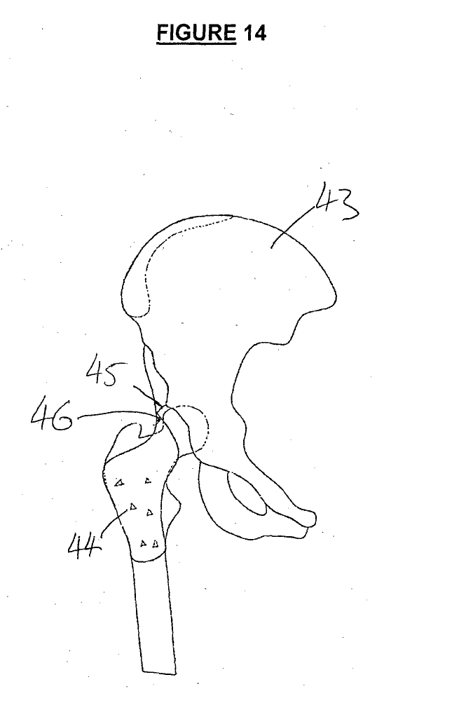

FIG. 15 is a partial view of a pelvis showing components of a prosthesis according to the present invention.

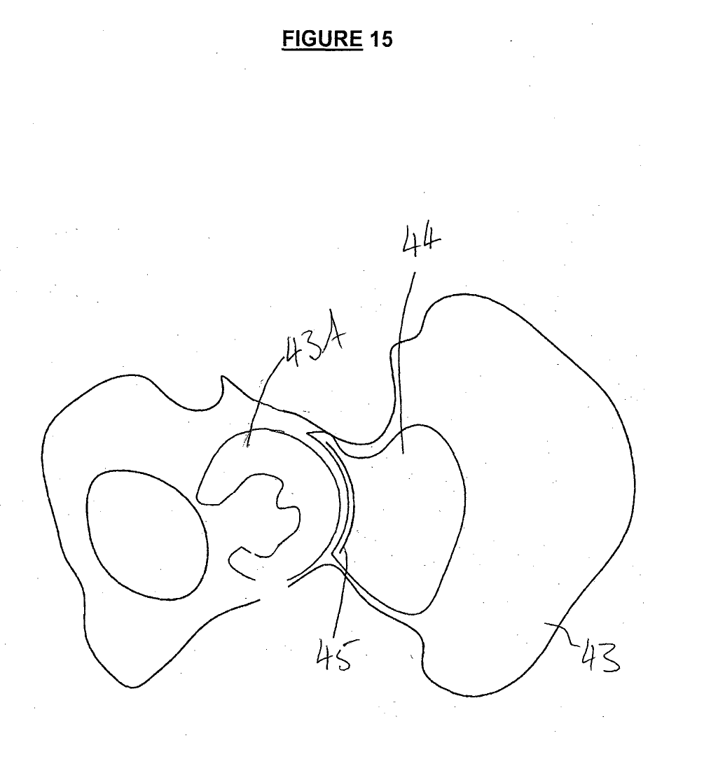

FIG. 16 is a front view of a shoulder joint including a prosthesis according to the present invention.

FIG. 17 is a side view of FIG. 16.

FIG. 18 is a side view of an ankle including a prosthesis according to the present invention.

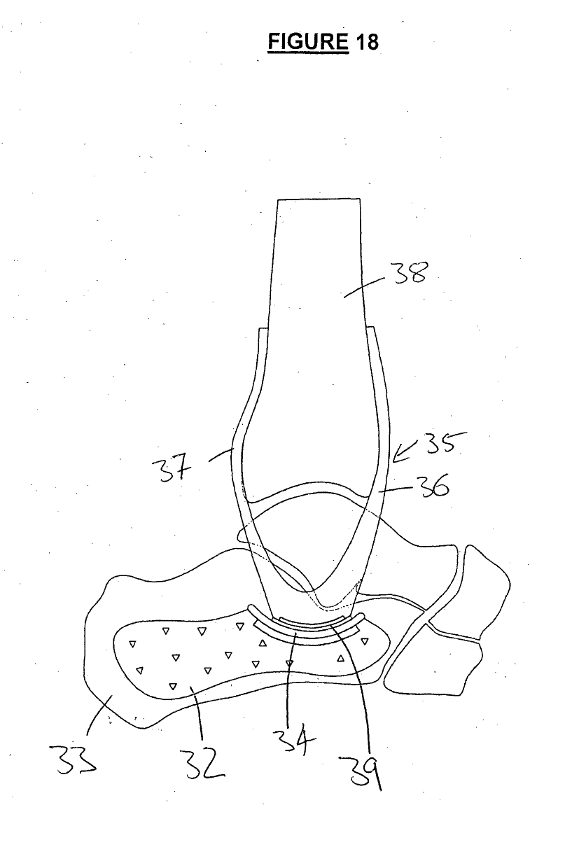

FIG. 19 is a back view of FIG. 18.

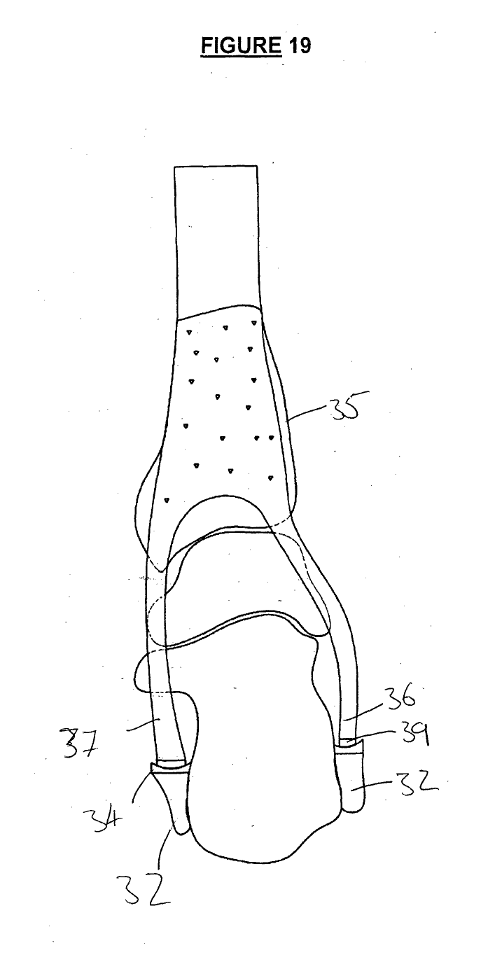

### BEST MODES FOR CARRYING OUT THE INVENTION

The prosthesis, methods of implantation and fixation will now be described in more detail with reference to the drawings. Specific discussion of the embodiment of the prosthesis in respect of a knee joint is provided below. Substantively similar principles apply to the components of the knee prosthesis as they do to prosthesis used in other joints such as the ankle, finger, elbow, or shoulder. One skilled in the art would appreciate that the discussion in respect of the knee prosthesis is equally applicable in respect of other joints. This is particularly so in respect of the fasteners and implantation method.

The present invention may be provided as a kitset of parts, including any one or more of the components described herein. It is envisaged that a kitset for an ankle, shoulder, elbow, finger or hip prosthesis could include components based on those described with reference to the knee prosthesis.

### Knee Prosthesis

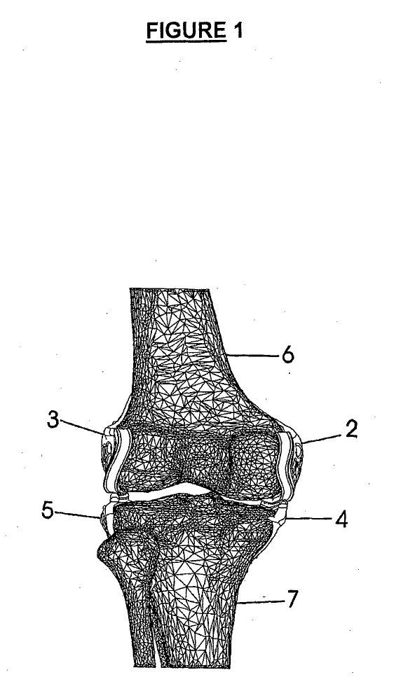

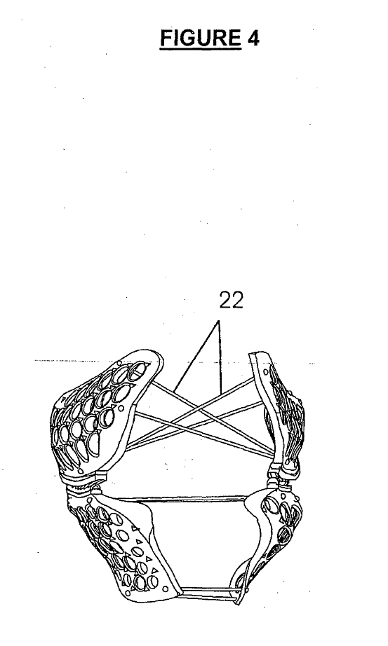

Referring specifically to FIGS. 1 to 9C, a preferred embodiment will be described in detail which utilises a pair of plates either side of the joint. However, it is to be appreciated that only one side of a joint could be treated with the present invention.

The prosthesis is provided for implantation into a knee joint and is attached directly to the outer surfaces of the distal femur and proximal tibia as generally illustrated in FIGS. 1-3. The prosthesis generally comprises a pair of femoral plates 2, 3 attached to the medial and lateral exterior surfaces of the distal femur respectively. Similarly, the tibial components of the prosthesis comprise tibial plates 4, 5 attached to the medial and lateral surfaces of the proximal tibia respectively. The femoral components 2,3 conform to the anterolateral/anteromedial surfaces of the distal femur end, approximately 5-7 cm of femur bone. The tibial components 4,5 conform to the anterolateral/anteromedial surfaces of the proximal approximate 5-7 cm of tibia.

The prosthesis is a low profile structure, being widest at the joint end and becomes progressively narrower further away from joint. The prosthesis allows space for important soft tissue structures including ligaments around the joint.

Each femoral and tibial component (2, 3, 4, 5) may have a different anatomical configuration depending on its position around the joint. i.e. each component may be configured according to the anatomical specificity of the bone involved insuring a good fit. Femoral components 2, 3 are attached to the anterolateral/anteromedial distal end of the femur, while the tibial components 4, 5 are attached to the anterolateral/anteromedial proximal end of the tibia.

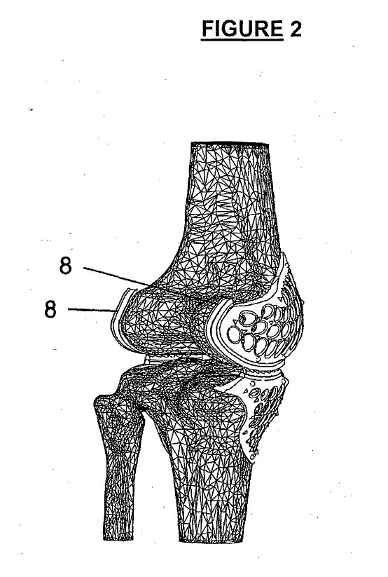

Articulating surfaces are provided on the distal margins of the femoral components 2,3 and on the proximal margins of the tibial components 4,5 to allow flexion and extension of the joint. The articulating surfaces of the femoral components approximately follow the lateral/medial borders of the native joint articulating surfaces respectively. As shown in FIG. 1 and FIG. 2 the articulating surfaces of the femoral components are shaped so that they provide a full range of flexion/extension for the knee joint. This should be become clearer from the following description.

The articulating surfaces are broader posteriorly to accommodate the rotation and sideway motion of the knee in flexion. The articulating surface is slightly concave to allow for the rotation and locking of the knee in the extension. Accordingly, a portion of the total load applied through the joint is carried by the prosthesis along the medial and lateral margins of the native joint structure.

In a preferred embodiment, a separate bearing surface 8 is attached to distal (and posterior) edge of the femoral plates 2, 3. It is also envisaged that the bearing surfaces could be formed integrally into the femoral plates 2, 3

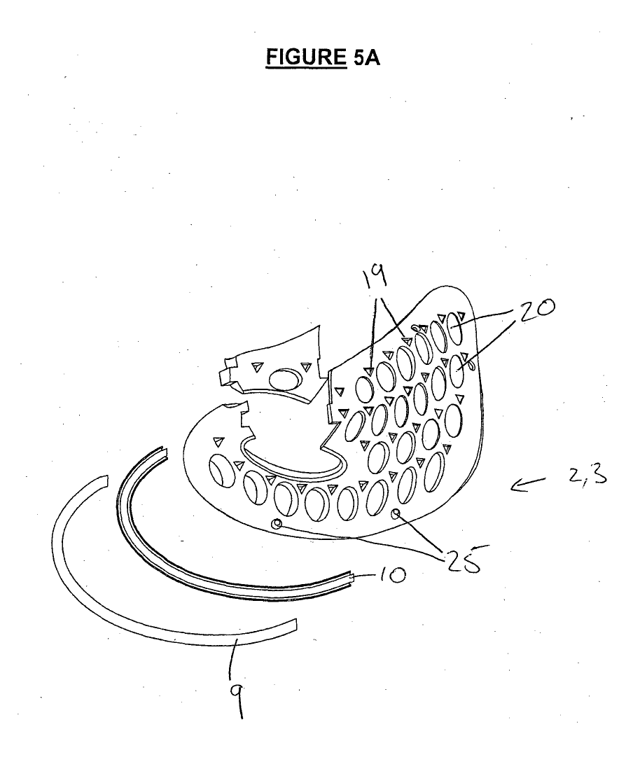

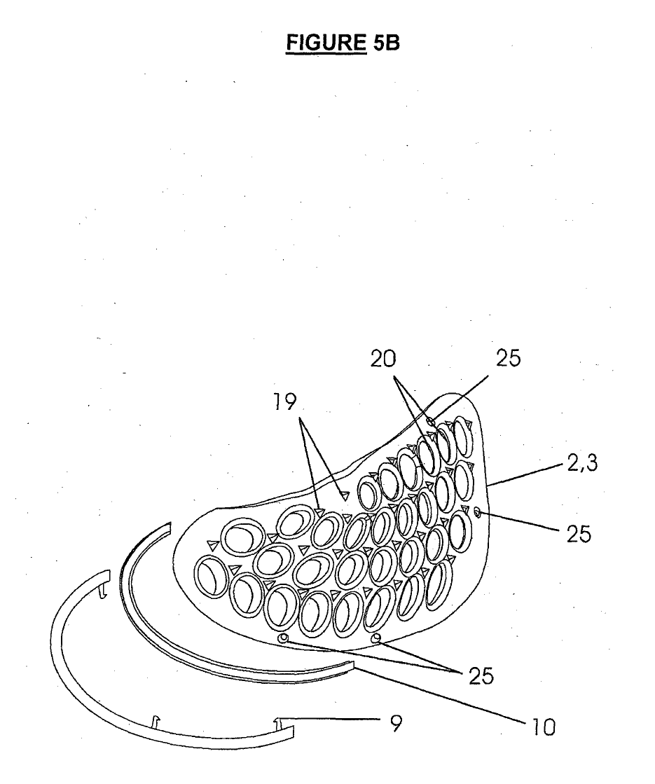

In a preferred embodiment, the femoral bearing surface 8 comprises a steel backed ultra high density ceramic material to improve its wear characteristics. The femoral bearing surface 8 is attached to the femoral component 2, 3 via fasteners. A first fastener embodiment is shown in FIG. 5A.

An alternate embodiment fastener system is shown in FIG. 5B in which hooks 9 which attach these two components.

The femoral bearing surface 8 preferably has dimensions of approximately 2-3 mm thickness and approximately 4-6 mm of width. The length of the bearing surface 8 can vary so as to fit different recipients. These are likely to be in the range of 12-18 mm.

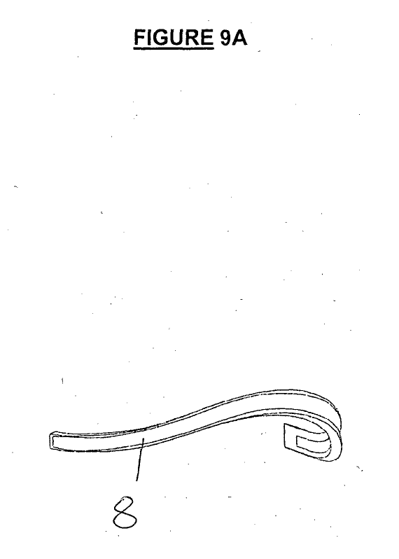

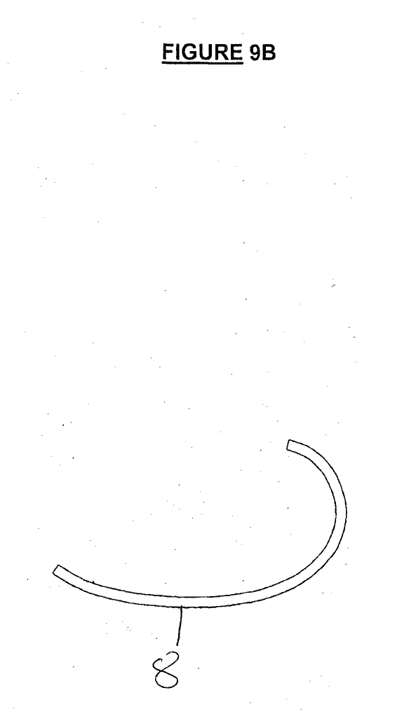

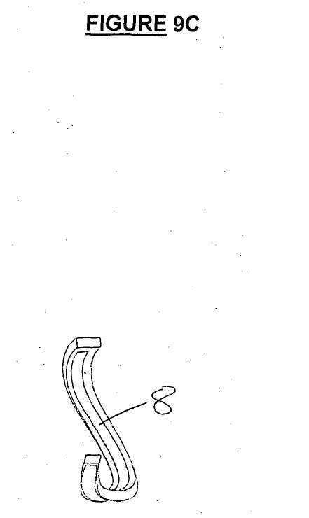

The femoral bearing surface 8 is shown in FIGS. 9A-9C and is shaped to correspond to and/or mimic the articulating surface of the native femur bone forming part of the knee. The bearing surface 8 does not have a simple mathematical shape. Rather, it is shaped and configured so as to mimic the function of and range of motion of, the native knee joint. This is important in the prosthesis providing a full range of motion being able to replace the knee joint rather than simply assist the native knee joint's operation.

In a preferred embodiment, a deformable component is provided in between the bearing surface 8, and femoral plate 2, 3. Preferably the deformable component 10 is approximately 2-3 mm thick and is made of a bio-compatible polymer, and operates to absorb some of the forces applied through the joint. Preferably, the polymer has a young's modulus of approximately 5-20 times that of articular cartilage and is substantially impervious to creep. In a most preferred embodiment the polymers modulus is 5-10 times that of cartilage. It is also preferred that the material have a Poisson ratio of approximately 0.3 which is typical of cancellous bone. For example, the deformable member 10 may be a synthetic carbon polymer (eg: PMMA). Preferably, the deformable component 10 is made from a material which will allow the joint (as a whole) to deform in a manner comparable to normal articular cartilage under the expected physiological stress. It may be preferable that the deformable component deforms slightly less than typical cartilage in order to increase the proportion of the load transferred by the prosthesis.

Other materials suitable for the deformable component are Ultra High Molecular Weight Polyethylene (UHMWPE); silicone polycarbonate urethane; or rotaxane.

The deformable component 10, provides a deformable structure between the comparatively rigid femoral component 2, and corresponding tibial component 4, and bone. The component 10 is preferably held in place between the bearing surface 8 and femoral plate 2.

The deformable component may be mated with the bearing surface 8 and femoral plate.

For instance, in the embodiment shown in FIG. 5A the femoral plate 2,3 may include a groove 17 which receives a corresponding projection or rib of the deformable component 10. Similar grooves and projections may be provided at the interface between the component 10 and the bearing surface 8.

In one embodiment, the tibial components are approximately 2-3 mm thick and approximately 4-6 mm wide.

Tibial articulating surfaces are provided on proximal margin of the tibial components 4,5. The tibial articulating surfaces are preferably shaped so as to correspond to and/or mimic approximately the middle two thirds of the medial/lateral border of the native knee joint articulating surface.

In one preferred embodiment, a separate bearing surface 11 may be secured to each of the tibial components 4,5. Each bearing surface has a shape that corresponds to the articulating surface of the native tibial component of the knee. The bearing surface 11 is a ceramic material secured to a metallic base. The ceramic material has a low frictional coefficient, therefore allowing the bearing surface 8 and bearing surface to slide across each other.

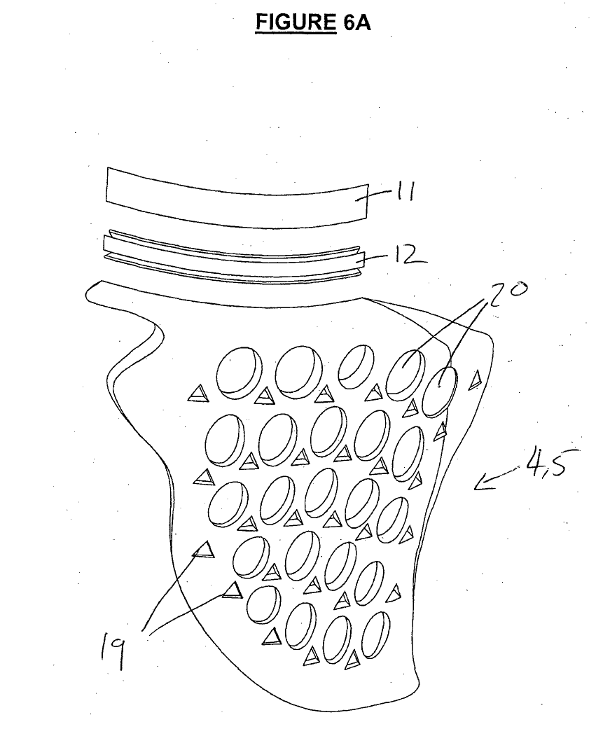

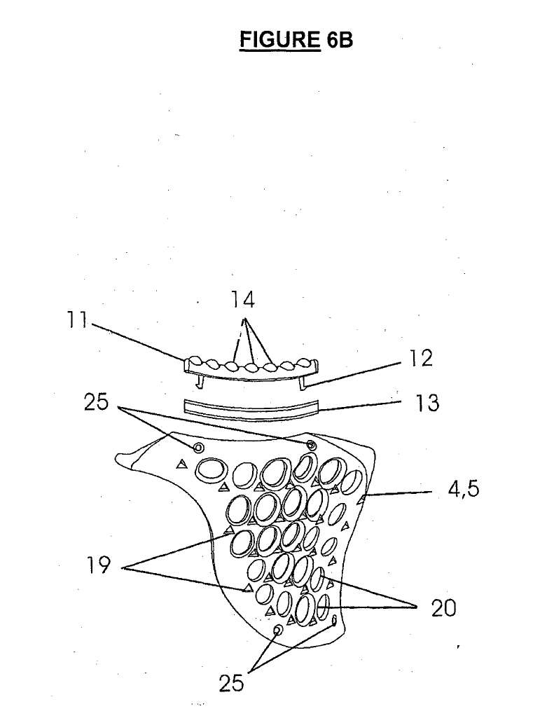

An alternative embodiment is shown in FIG. 6B, where the bearing surface 11 includes a plurality of ball bearings. The balls 14 are approximately 2 mm in diameter and made from ceramic coated stainless steel. There may be approximately 5 to 10 bearings 14, and the corresponding bearing housing may also be coated with a ceramic material to improve the wear characteristics of the interface. Alternatively, the tibial bearing surface may be a polished metal or ceramic surface.

The bearing surface 11 acts as an articulating surface so as to provide a range of motion for the joint.

The bearing surface 11 can be secured to the tibial component using several fastener arrangements. For instance, one particularly preferred embodiment is shown in FIG. 6A, where a latch on bearing service 11 mates with a corresponding groove (not shown) on tibial component 4,5 to secure these together.

An alternate securing system is shown in FIG. 6B. A number of hooks 12 engage with apertures in tibial plate 4,5.

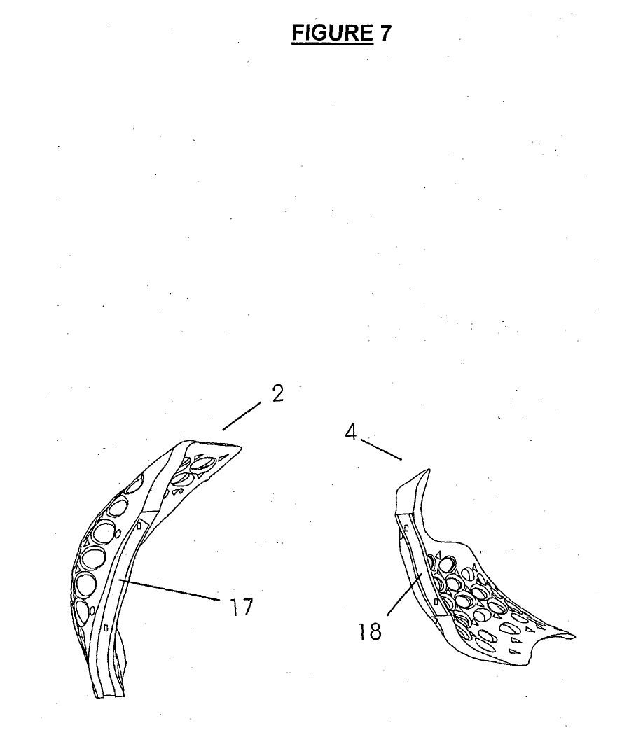

In between the bearing surface 11, and the tibial plate 4,5, is a deformable component 13 which is approximately 2-3 mm thick. To secure the deformable component 13, between the bearing surface and the tibial component, the underside of bearing surface 11 may have a longitudinal groove (shown in FIG. 7) for receiving and mating with the deformable component 13. Similarly, the deformable component may have a rib or projection on the underside for a corresponding groove 18 in the proximal edge of the tibial component 4,5.

In their preferred forms, the femoral components 2,3 and tibial components 4,5 are made from a non-bioactive material that is stiff and hard such as stainless steel or titanium. In a most preferred form, titanium is used. It is also preferred that both the femoral and tibial components include a number of apertures or holes 20. This reduces the bulk of the metal without significantly compromising its stress distributing properties. The holes may also allow soft tissue attachment and hence the nutrition of the bone thereby not disturbing normal biology significantly.

The outer surface of the femoral and tibial components 2-5 are preferably polished to minimize rubbing of the surrounding soft tissues which may result in irritation. The inner surfaces are also smooth, but may include multiple protrusions (not shown) to keep the component distanced from the bone surface. For example, a number of spaced protrusions approximately 1 mm long may project from the inner surfaces, to separate the plates from the bone, in order to reduce the risk of pressure necrosis of the bone commonly seen after plating of fractured bone.

The prosthesis according to the present invention is a stress sharing device suitable for minimally invasive, surgical implantation around the knee joint without compromising the native joint surface. Accordingly, it substantively transfers potentially damaging stress from the joint and distributes this to the tibia and femur bones at locations away from the joint. This allows the joint to repair itself by maintaining the basic physiological strain at the joint surface. In extreme cases the prosthesis could take substantially all the stress from the joint. The bearing surfaces facilitate the joint having a desired range of motion.

The present invention may also find application as a stabilisation method for intra-articular fracture.

### Fastening Methods

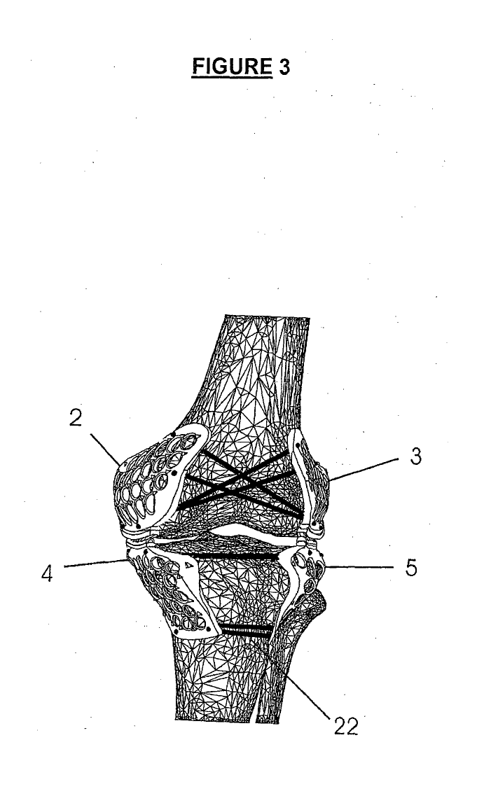

In one preferred form, the tibial and femoral components 2-5, have multiple triangular holes 19 to accommodate corresponding bone fasteners 15. Preferably, the flat portion of the triangle shaped holes 19 is oriented to be perpendicular to the line of stress through the joint, to improve transmission of stress from the prosthesis to the bone. That is, the points of the triangle are oriented to point towards the respective joint surface. As best shown in FIG. 3, the triangle points face downwards for the femoral components 2,3, and triangle points face upwards for the tibial components 4,5.

The bone fastener pins 15 are designed to transmit the stress from the femoral and tibial plates to the corresponding bony structures to which they are attached. The fastener 15 is hammered into the bone through the apertures 19 in the femoral and tibial components.

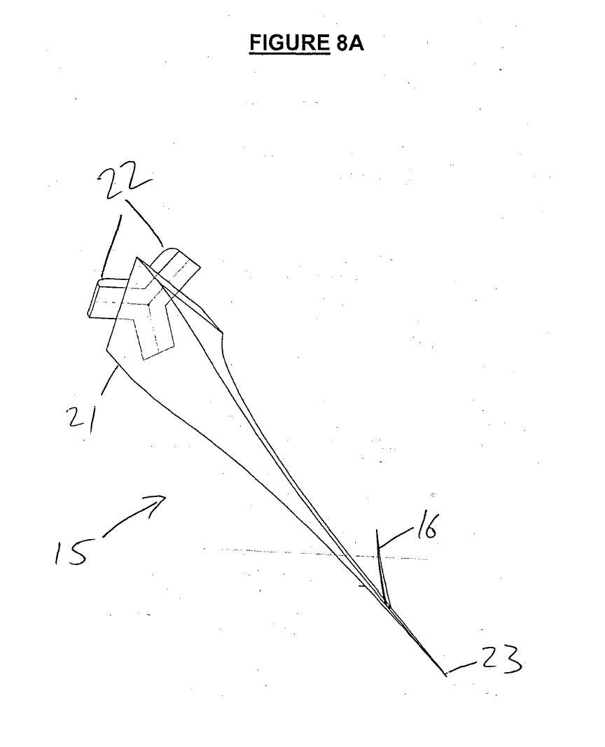

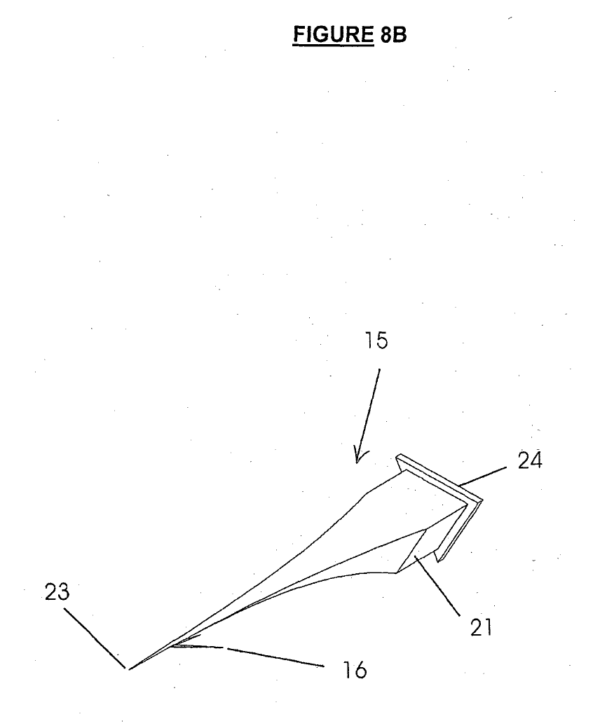

One embodiment of a fastener is shown in FIG. 8A. One end of the fastener 15 includes a head 21, having a triangular cross section. The head 21 narrows through body section 22 to point 23. End 21 has an engagement point 22 and a plurality of barbs 16 extend from body section.

In use, fastener pins 15 are inserted through holes 19 in the plates 2-5 further than required. Engagement point 22 is used to draw the fastener pin 15 backwards towards plate 2-5. This assists in barbs 16 engaging the cancellous bone so as to secure the fastener pins 15, and thereby the plates 2-5, in position.

An alternate embodiment of the fastener pin is shown in FIG. 8B. End 21 has a thin sheet of elastic metal 24 attached at the center. Elastic metal sheet 24 is larger in size as compared to end 21. During insertion, end 21 should be pushed in further to preload the fasteners pins in their inserted position. This will create elastic recoil and help to fix the barb end in the cancellous bone. The point end 23, preferably includes a barb 16 for fixation in the cancellous bone. In its preferred form, the barb 16 is approximately 5 mm in length.

The bone fastener may be made from stainless steel, or most preferably, titanium.

The medial and lateral femoral plates 2,3 may also be fixed with a number of locking rods 22 as shown in FIGS. 3 & 4. These four locking rods 22 pass through the corresponding bone are fix the pair of femoral plates and tibial plates together respectively. The plates 2-5 also have holes for the attachment of the cross rods 22. In order to insert the rods 22, a hole is drilled in the bone to accommodate the rods. A guide is used to direct the drill hole between the corresponding holes 25 in the femoral/tibial plate. The required length of the rods 22 are measured by the guide. A ball tipped rod 22 is inserted from one side, while the other end of the rod 22 is threaded. A nut is applied to the threaded end, and tightened to achieve the required strain. The excess thread can then be cut flush.

Alternatively, it is envisaged that conventional screwing techniques could also be used to fix the prosthesis to the bony structures.

### Implantation Method

A method is provided for implantation of the prosthesis according to the present invention. The method will be described herein with reference to insertion of the knee joint. However this should not be seen as limiting and it should be appreciated that similar steps are involved in implanting prosthesis to other joints.

One skilled in the art should be able to extrapolate from the steps described herein so as to work the present invention.

Before implanting the prosthesis, the patient's knee joint may be examined by a non-invasive imaging procedure such that appropriately sized and shaped components may be selected. A variety of non-invasive imaging devices may be suitable, for example CT scan, or X-ray devices and the like. Two methods of non-invasive imaging for selection of a suitable prosthesis are preferred.

In the first method, CT scan or other non-invasive imaging scans, optionally coupled with exterior measurements of the dimensions of the relevant proximal tibia and proximal femur bone, may be used to establish a library of prostheses whose size and geometry differ according size of the patient. A limited number of “standard” prostheses are then made to meet the requirements of a generic population of patients. In this first method, a non-invasive imaging scan, such as an X-ray or CT scan, together with clinical measurement will enable the surgeon to select a prosthesis of the best size and shape from the library for a particular patient. With this method, it is expected that some modification of the patients bony structure may be necessary. However, an extensive set of standard sizes can be created to minimize the modification required to the joint's anatomy.

In a second method, each patient receives one or more prostheses that are custom tailored for the individual. Such a prosthesis may be constructed from imaging data (i.e., X-ray or CT scan data) by a suitable computer program. The second method is likely to result in an improved fit to a patient's unique anatomy, and/or reduce the need to shape the exterior surfaces of the patient's bones.

Surgery can be done under general anaesthesia or regional anaesthesia. The patient is positioned supine with radiolucent wedge located underneath the knee and the operation is done under tourniquet control and image intensifier guidance.

An anterolateral and posteromedial approach to the distal femur and proximal tibia is utilized to approach the distal femur and proximal femur. All the soft tissue is taken off from the bone as a soft tissue sleeve.

Insertion of the prosthesis of the present invention is typically done via a 10 cm to 14 cm medial and lateral incision. The articulating body of the femoral component is aligned with the lateral/medial edge of joint surface. Its position is checked visually and radiological using intra-operative X-ray. Once acceptable alignment is achieved, it is temporarily fixed with the help of wires. A set of standard size templates may be provided during the surgery to achieve initial alignment and appropriate sizing. Once an exact size is determined the prosthesis is applied using the initial temporary wires. All other components of the prosthesis are attached on this base line.

The tibial plate component articulates with femoral articulating body and it can be preloaded depending on the clinical requirement. A pre-compression of the polymer insert 10 can function to take the resting stress from the joint surface.

The first step of implantation is to align an appropriately sized articulating body of the femur with the joint surface and the lateral/medial edge of the femoral condyle.

The implant should correspond to the condylar line in a lateral knee X-ray. Next, the tibial articulating body is placed opposing the femoral plate. Both plates are temporarily fixed with K-wires. The appropriate position and size can be checked using an image intensifier and an AP view is taken to check the joint space. A pre-stress device can be used to pre-stress the implant according to clinical requirement by compressing the deformable component. Once in the correct position, rods 22 are used to fix the plates, and the triangular fasteners are hammered into the bone. The temporary K-wires are then removed. The joint can then be tested and taken through the full range of motion.

### Elbow Prosthesis

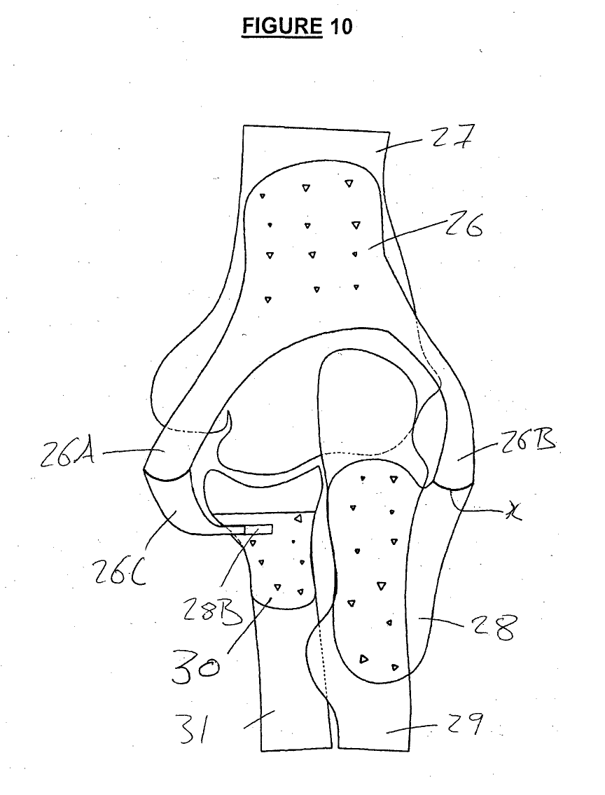

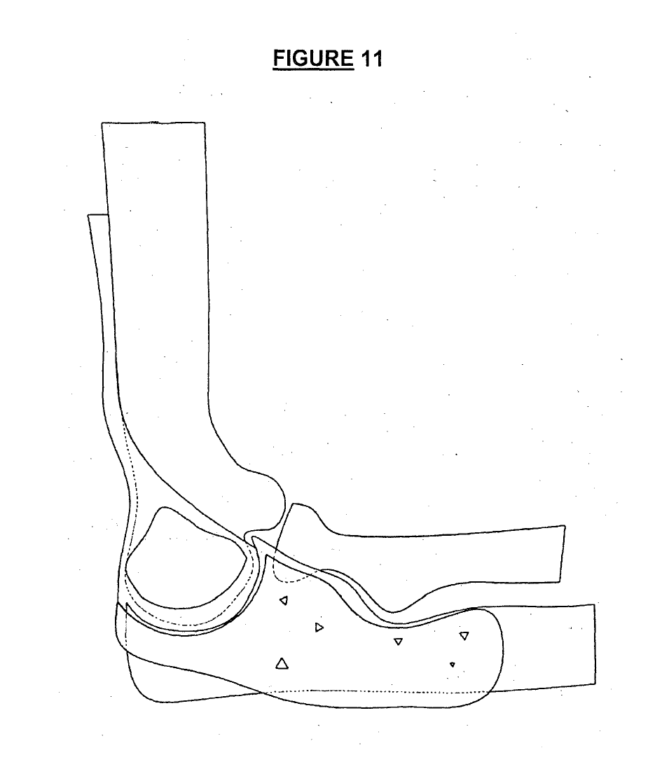

Referring now to FIGS. 10 and 11 showing an elbow having a prosthesis according to the present invention in extension and flexion.

A first plate 26 is secured to distal end of humerus 27. The first plate has a general “Y” shape with first arm 26A and second arm 26B. The arms diverge so as to surround the condyle of humerus 27.

A protrusion 26C provides a bearing surface between first plate 26 and third plate. A second plate 28 is secured to proximal end of ulna 29 and a third plate 30 is secured to proximal end of radius 31.

Arm 26B provides a bearing surface that cooperates with bearing surface on second plate (indicated generally as X) so as to facilitate the ulna moving with respect to the humerus and to provide for flexion and extension of the elbow joint. This is achieved by the bearing surfaces being shaped so as to correspond to and/or mimic the articulating surfaces of a native elbow joint responsible for flexion and extension.

Protrusion 26C acts as another bearing surface by slidingly cooperating with groove 30B in the third plate. The protrusion 26C can slide across arm 26A. This provides rotational motion of the radius with respect to the humerus. That is, cooperation between bearing surfaces on the first and third plates facilitates pronation and supirnation of the radius 31.

### Ankle Prosthesis

The ankle prosthesis is configured to replicate motion of the native ankle joint. This is in contrast to embodiments such as the knee prosthesis or elbow prosthesis which have bearing surfaces configured to replicate the knee joint shape. This is necessary as the configuration of a foot and ankle joint means that there is little room to secure components of the prosthesis.

A first plate 32 having a bearing surface 33 is secured to calcaneum bone 34 on the lateral/medial edge of a foot. Bearing surface 33 has a generally concave shape when viewed from the lateral edge of the foot.

A second plate 35 has first arm 36 and second arm 37. The arms 36, 37 diverge so as to be able to surround front and back edges of tibia 38. The arms provide bearing surfaces 39 which are generally convex in shape when viewed from the lateral edge of the foot.

Bearing surfaces 33, 39 are arcs of a circle. Therefore, the bearing surfaces define a range of motion similar to the native ankle joint. However, bearing surfaces 33,39 are not shaped to correspond to the native ankle joint articulating surface.

Bearing surface 33 is slightly wider laterally than bearing surface 39. This allows for the lateral movement of the foot.

### Finger Prosthesis

A finger prosthesis is configured and arranged in a similar manner to a knee prosthesis according to the present invention. That is, a first plate 40 is attached to a distal portion of a bone forming part of a joint, and a second plate 41 is attached to a proximal part of a bone forming part of the joint. The first and second plates 40, 41 have bearing surfaces (indicated generally by Y) that cooperate to guide the second bone through a desired range of motion.

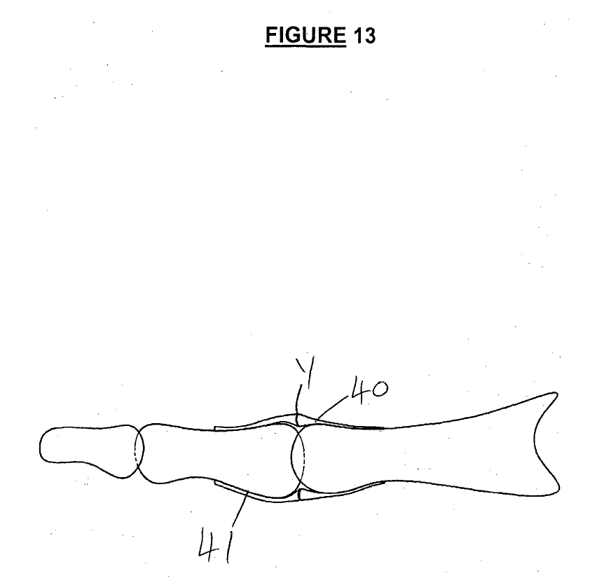

It is possible to have pairs of plates on distal sides of a joint as is shown in FIG. 13.

The shape and configuration of the plates and their respective bearing surfaces will vary according to the finger joint within which the prosthesis is used. For instance, different shapes and ranges of motion are needed in a finger joint between a metacarpal and a proximal phalanges, compared to a finger joint between proximal phalanx and middle phalanx.

As with other embodiments, the prosthesis guides the bones forming the joint through a range of motion and acts as a surface for that motion to occur.

Deformable components may or may not be used with a prosthesis for a finger joint as these do not experience the same stresses as do load bearing joints such as the knee or ankle.

### Hip Prosthesis

A hip prosthesis includes a first plate 42 secured to pelvis 43 near acetabulum 43A. First plate 42 has a bearing surface in the form of a socket having a curve. The socket has a lip which extends away from the pelvis so as to define a cavity to receive a corresponding bearing surface 45.

A second plate 44 is secured to proximal end of femur. Second plate has a bearing surface 45 with the same curvature as that of first plate's bearing surface 46. However bearing surface 45 is smaller then bearing surface 46. This will allow bearing surfaces 45,46 to move with respect to each other, and therefore provide a range of motion for the hip joint.

Bearing surface 46 extends over the edge between the native femur and acetabulum so as to engage with bearing surface 45.

The radius of curvature of the bearing surfaces 45,46 is greater than the radius of curvature of the articulating surfaces in the native hip joint. This may assist in keeping the native joints separated from each other.

The bearing surfaces 45,46 guide the femur bone and facilitate this moving with respect to the hip joint. The components maintain separation between the bones of the hip joint and provide a surface for relative movement of these.

A deformable component (not visible) can be used between the first and second plates. This allows forces applied to the joint to be transferred into the cartilage of the joint. However the deformable component is configured so as to maintain separation of the hip bones, so that movement of the femur with respect to the pelvis occurs via the bearing surfaces.

### Shoulder Prosthesis

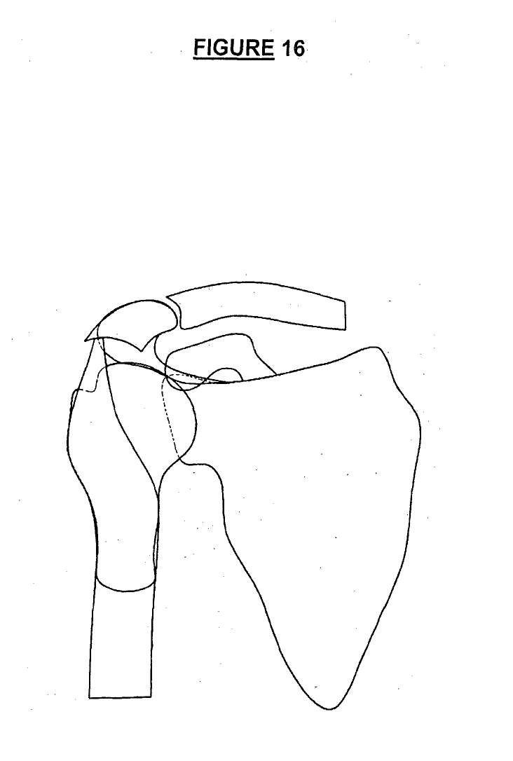

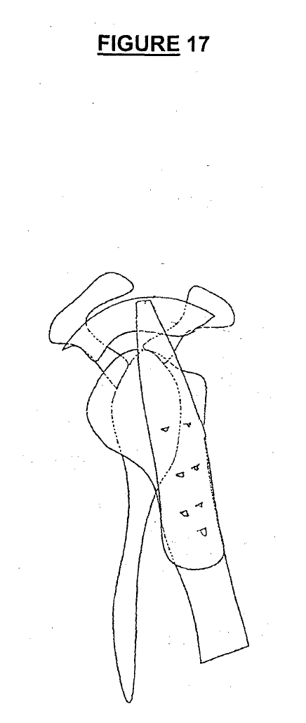

A shoulder prosthesis according to the present invention is shown in FIGS. 16 and 17. A first plate is attached to the top of scapula at the lateral margin. The first plate provides a bearing surface in the form of a recess. A lip extends back over recess to provide a cavity having a curvature. However, the radius of curvature of the first plate's bearing surface is slightly greater than the radius of the native glenoid fossa.

A second plate 48 is attached to proximal end of humerus. The second plate provides a bearing surface which extends up and over the top of outside edge of humerus condyle. The second plate's bearing surface has the same radius of curvature as the first plate's bearing surface but is smaller. This allows the second plate's bearing surface to move with respect to the first palte.

The bearing surfaces cooperate so as to provide a range of motion for the prosthesis.

The radius of curvature of the bearing surfaces is slightly greater than the radius of curvature of the articulating surfaces of the native shoulder joint. This pushes the humerus out laterally with respect to the scapula so as to ensure that the motion of the joint occurs on the bearing surfaces, rather than the articulating surfaces of the native joint. This may assist in keeping the native joints separated from each other.

While embodiments of the invention have been illustrated and described, it is not intended that these embodiments illustrate and describe all possible forms of the invention. The words used in the specification are words of description rather than limitation, and it is understood that various changes may be made without departing from the spirit and scope of the invention.

Aspects of the present invention have been described by way of example only and it should be appreciated that modifications and additions may be made thereto without departing from the scope thereof as defined in the appended claims.

Prosthesis claims

What I claim is:

1. According to one aspect of the present invention, there is provided a prosthesis for insertion into a joint, including
a first plate configured for fixing to a first bone forming part of the joint,
a second plate configured for fixing to a second bone forming part of the joint,
bearing surfaces associated with the first plate and second plate,
characterised in that
the bearing surfaces are shaped to cooperate with each other to guide the movement of the second bone relative to the first bone through a desired range of motion.

2. A prosthesis as claimed in claim 1, where the bearing surfaces are shaped so as to provide a range of motion corresponding to the native joint.

3. A prosthesis as claimed in claim 2, wherein the plates are aligned so that the bearing surfaces remain in contact throughout full flexion and extension forming the range of motion of the joint.

4. A prosthesis as claimed in any one of the preceding claims, wherein a pair of first plates are fixed on the lateral and medial margins of a joint respectively, and a pair of second plates are fixed on the lateral and medial margins of a joint respectively.

5. A prosthesis as claimed in any one of the preceding claims, wherein the bearing surface associated with the second plate includes a plurality of ball bearings arranged along its length.

6. A prosthesis as claimed in any one of the preceding claims, including a deformable component configured to transfer a portion of force applied through the joint into cartilage in the joint.

7. A prosthesis as claimed in claim 6, wherein the deformable component has material properties such that it deforms less than or equal to the joints native cartilage.

8. A prosthesis as claimed in claim 6, wherein the deformable component has material properties such that it is 5-20 times as stiff as typical articular cartilage.

9. A prosthesis as claimed in any one of the preceding claims, wherein the bearing surface associated with said first plate is a polished ceramic material.

10. A prosthesis as claimed in any one of the preceding claims, wherein the first and said second pairs of plates are shaped to correspond to the bones of a joint.

11. A prosthesis as claimed in any one of the preceding claims, wherein the first and second plates include a plurality of apertures.

12. A prosthesis as claimed in claim 9, wherein at least some of the apertures are generally triangular is shape.

13. A prosthesis as claimed in claim 12, wherein the point of the triangular apertures are aligned to face toward the joint surface.

14. A prosthesis as claimed in any one of claims 4 to 13, wherein a plurality of fastening rods extend across the joint between each of said first plates.

15. A prosthesis as claimed in any one of claims 1 to 14, configured for insertion into a knee.

16. A prosthesis as claimed in any one of claims 1 to 14, configured for insertion into an ankle.

17. A prosthesis as claimed in any one of claims 1 to 14, configured to insertion into a shoulder.

18. A prosthesis as claimed in any one of claims 1 to 14, configured for insertion into a hip.

19. A prosthesis as claimed in any one of claims 1 to 14, configured for insertion into a finger.

20. A fastener for fixing a prosthetic structure to bone, including
a body with a length,
a first point end,
a second end distal to the first end
wherein the second end is configured to facilitate inserting the fastener into bone
characterised in that
the body has a section with a generally triangular cross section.

21. A fastener as claimed in claim 20, wherein the shaft includes at least one barb.

22. A fastener as claimed in claim 20 or claim 21, wherein the second end of the shaft includes a substantially straight shoulder portion and tapers from said shoulder portion to said point.

23. A fastener as claimed in any one of claims 20 to 22, wherein the head is deformable and provides some elastic recoil to the fastener after being inserted deep enough to deform said head.

24. A fastener as claimed in any one of claims 20 to 23, wherein the second provides an engagement point to enable the fastener to be moved with respect to a bone so as to cause the barb to engage the bone.
Surgical Method claims

25. A method of implanting a prosthesis in a joint, including the following steps in any order:
(a) aligning a first plate including a bearing surface with a first bone forming part of the joint;
(b) securing the first plate to the first bone;
(c) securing the second plate to the second bone;
(d) aligning a second plate including a bearing surface with a second bone forming part of the joint;
wherein the respective bearing surfaces of the first plate and the second plate contact;
characterised by the step of:
(e) configuring the bearing surfaces to cooperate so as to guide the first and second bones to move through a desired range of motion.

26. A method of implanting a prosthesis as claimed in claim 25, wherein before the steps of fixing of the plates, the plates are temporarily fixed and the correct positioning is confirmed by imaging.

27. A method of implanting a prosthesis as claimed in either one of claim 25 or 26, including the step of pre-loading the bearing surfaces before the step of fixing.

28. A method of implanting a prosthesis as claimed in any one of claims 25 to 27, wherein the plates are fixed with fasteners according to any one of claims 15 to 19.

29. A method of implanting a prosthesis as claimed in any one of claims 25 to 28, wherein the steps are repeated for both sides of the knee joint.

30. A method of implanting a prosthesis as claimed in claim 29, wherein the step of fixing further includes inserting a plurality of fastening rods extending across the joint between each of said first plates and second plates.

31. A method of fastening a prosthesis, including:
fastening a prosthetic device to bone using the fastener of any one of claims 25 to 30.

32. A method as claimed in anyone of claims 25 to 31, wherein the prosthesis is inserted into a knee.

33. A method of treating osteoarthritis characterised by implementing the method claimed in any one of claims 25 to 32.
“Kit of parts” claims

34. A prosthesis kit, including:
(a) a first plate configured for fixing to a first bone forming part of a joint;
(b) a second plate configured for fixing to a second bone forming part of the joint;
wherein each of the first plate and second plate have bearing surfaces;
characterised in that bearing surfaces are shaped to co-operate with each other when the prosthesis is fitted into a joint so as to guide the movement of the second bone relative to the first bone through a desired range of motion.

35. The prosthesis kit as claimed in claim 34, wherein the second bearing surface includes a plurality of ball bearings arranged along its length.

36. A prosthesis kit as claimed in either one of claim 34 or 35, including first and second deformable components.

37. The kit as claimed in claim 36, wherein the deformable components have material properties such that they deform less than or equal to knee joint cartilage under typical physiological loads.

38. A prosthesis kit as claimed in either one of claim 35 or 36, wherein first and second deformable components have material properties such that they are 5-20 times as stiff as typical articular cartilage.

39. A prosthesis kit as claimed in any one of claims 35 to 38, wherein the first bearing surface is a polished ceramic material.

40. A prosthesis kit as claimed in any one of claims 35 to 39, wherein said first or said second bearing surface includes a plurality of fasteners adapted to secure the bearing surface to the plate.

41. A prosthesis kit as claimed in any one of claims 35 to 40, wherein said kit includes a pair of the first plates and said second plates,
a pair of the first and the second bearing surfaces, and
a pair of first and second deformable components.

42. A prosthesis kit as claimed in any one of claims 28 to 35, wherein the kit further includes a plurality of fasteners.

43. A prosthesis for implantation into a joint substantially as herein described and with reference to any one or more of the drawings.

44. A fastener for fixing a prosthetic structure to bone substantially as herein described and with reference to any one or more of the drawings.

45. A method of implanting a prosthesis substantially as herein described and with reference to any one or more of the drawings.

46. A method of fastening a prosthesis substantially as herein described and with reference to any one or more of the drawings.

47. A prosthesis kit comprising any one or more of the parts substantially as herein described and with reference to any one or more of the drawings.

48. A method of treating osteoarthritis substantially as herein described and with reference to any one or more of the drawings.
